(19) United States
(12) Patent Application Publication
(10) Pub. No.: US 2011/0172591 A1
(43) Pub. Date: Jul. 14, 2011

(54) PORTABLE TOPICAL HYPERBARIC SKIN THERAPY AND WOUND TREATMENT SYSTEM

(75) Inventor: Eilaz Babaev, Minnetonka, MN (US)

(73) Assignee: BACOUSTICS LLC, Minnetonka, MN (US)

(21) Appl. No.: 12/686,419

(22) Filed: Jan. 13, 2010

(51) Int. Cl. A61M 35/00; A61K 9/14; A61K 33/00; A61P 17/02

(52) U.S. Cl. 604/24; 424/484; 424/613

(57) ABSTRACT

The invention discloses methods and devices for promotion of skin healing, skin conditioning and skin rejuvenation for skin and tissue repair. More specifically, the invention is directed toward the manufacture of a storable therapeutic pad containing a therapeutic solution within a matrix. The therapeutic solution is produced by mixing oxygen with a fluid such as normal saline using an ultrasound station. Alternatively, the matrix may be composed of gels, solids and fibrous materials. The ultrasound station can be used to form micro-bubbles containing oxygen which allow storage of relatively high quantities of oxygen in a storage container containing the therapeutic pad. Micro-bubbles may be attached to stands or contained within a closed-cell foam. At the appropriate time, the storage container is opened and the therapeutic pad is attached to the wound site. The therapeutic pad is held between an impermeable layer and the wound site with an adhesive layer.

### FIELD OF THE INVENTION

The present invention relates to the promotion of treating skin to promote healing of skin tissue. More particularly, the present invention relates to devices and methods of ultrasonic application of oxygen into a therapeutic solution which is infused into a therapeutic pad. The therapeutic pad may be of a liquid, gel, solid of fibrous character. The therapeutic pad is storable for later use in topical oxygen therapy to a wound site to promote the healing of skin tissue.

### BACKGROUND OF THE INVENTION

It is known that providing a supply of oxygen to a wound to or through the skin (e.g., ulcers, abrasions, cuts, sores, etc.) promotes skin conditioning, skin rejuvenation and healing of wounded skin tissue. Oxygen from a blood supply is of course often disrupted by a wound injury due to vascular disruption and blood clotting at the wound site. Oxygen therapy is used for inducing the growth of new skin tissue to close and heal ischemic wounds. Supplying oxygen to a wound on a continuous and ambulatory basis is of benefit to speed healing and reduce infection. The oxygen dressing described below can be complimentary to other therapies and can address a rate-limiting step for various types of wounds.

Topical oxygen therapy calls for applying oxygen directly to an open wound. The oxygen dissolves in tissue fluids and improves the oxygen content of the intercellular fluids. Injuries and disorders which may be treated with topical oxygen include skin conditioning, skin rejuvenation, necrotizing fasciitis, pyoderma gangrenosum, refractory ulcers, diabetic foot ulcers and decubitus ulcers (bed sores) as well as cuts, abrasions, and surgically induced wounds or incisions.

The treatment of open wounds that are too large to spontaneously close has long been a troublesome area of medical practice. Closure of an open wound requires inward migration of surrounding epithelial and subcutaneous tissue. Some wounds, however, are sufficiently large or infected that they are unable to heal spontaneously. In such instances, a zone of stasis in which localized edema restricts the flow of blood to the epithelial and subcutaneous tissue forms near the surface of the wound. Without sufficient blood flow, the wound is unable to successfully fight bacterial infection and is accordingly unable to close spontaneously.

An initial stage of wound healing is characterized by the formation of granulation tissue which is a matrix of collagen, fibronectin, and hyaluronic acid carrying macrophages, fibroblasts, and neovasculature that forms the basis for subsequent epithelialization of the wound. Infection and poor vascularization hinder the formation of granulation tissue within wounded tissue, thereby inhibiting wound healing. It therefore becomes desirable to provide a technique for increasing blood circulation within wounded tissue to promote spontaneous healing and to reduce infection.

Poor blood circulation and infection at the wound may also hinder attachment of skin grafts or flaps upon wounded tissue. Skin grafts and flaps will not attach to tissue that is poorly vascularized, infected or necrotic. However, grafts and flaps can be used with much greater success on tissue that, although wounded, is able to form granulation tissue. Accordingly, a technique for promoting blood circulation at the wounded tissue would also promote successful attachment, or “take,” of skin grafts or flaps to the wounded tissue as a consequence of increased blood circulation within the grafts or flaps.

In light of the documented benefits of such oxygen therapy, there have been several proposed methods for providing such an oxygen supply to a wound or regulating the oxygen concentration in the vicinity of a wound while also preventing contamination of the oxygen supply from the wound. Prior art teaches the application of topical hyperbaric oxygen by placing the entire affected limb of a person in a sealed chamber that features controlled pressure sealing and automatic oxygen regulation control. Not only are such oxygen chambers expensive and difficult to sterilize, however, they are also cumbersome in that the chamber must be hooked up to an external oxygen tank, limiting the patient's mobility. In addition, because the entire limb is placed in a chamber or bag, large areas of skin may be unnecessarily subjected to high levels of oxygen. Such high levels of oxygen present risks of vasoconstriction, toxicity and tissue destruction. Another disadvantage of these units is that they only deliver vaporized or nebulized medications.

U.S. Pat. Nos. 5,578,022, 5,788,682, and 7,160,553 describe systems in which oxygen producing devices are incorporated into a patch or bandage which is placed directly over a wound. These devices tend to be cumbersome, expensive and have undesirable side reactions.

Therefore, a need exists for a convenient and inexpensive means of routinely treating patients having surface wounds of varying size, shape, and severity. Variations in wound type and other patient indications dictate variations in desired medications for treatment, such as antibiotics, growth factors, enzymes, hormones, insulin, anesthetics, and the like.

### SUMMARY OF THE INVENTION

The present invention is directed towards apparatus and methods for promotion of skin conditioning, skin rejuvenation and healing of wounded skin tissue. More specifically, the invention is directed toward the manufacture of a storable therapeutic pad containing a therapeutic solution within a matrix. The therapeutic solution is produced by mixing oxygen with a fluid such as normal saline using an ultrasound station. The use of gels, solids and fibrous matrices are also disclosed.

Oxygen is essential for many important aspects of the skin conditioning or healing process. For example, oxygen is required for cellular respiration, the process by which cells produce the energy needed to repair the wound. Oxygen is generally supplied to tissues of the body through the body's circulation system. Unfortunately, the blood supply to wounded tissue is often diminished or compromised. Consequently, the amount of oxygen reaching wounded tissue is often reduced. Not only can reduced oxygen levels inhibit the ability of cells to produce energy and/or heal a wound, reduced oxygen levels can lead to the production of an anaerobic environment within the wound favoring the development of certain infections. When treating wounded tissue, infected or otherwise, oxygen may be indirectly delivered to the tissue via diffusion by placing the wound in an oxygen rich environment or placing an oxygen releasing compound over the wound.

An ultrasound station provides rapid saturation and even super-saturation of the fluid by thorough mixing and production of micro-bubbles within a matrix. The matrix may be provided in a variety of configurations including a liquid, gel, solid, stranded or combination of these. In any event, the matrix is preferably a multi-phase material with oxygen dissolved as well as suspended oxygen micro-bubbles within the basic matrix structure. The matrix is typically constructed into a therapeutic pad and placed in a storage container. The therapeutic pad may be stored until needed in a suitable storage container to maintain high oxygen levels within the matrix. The stored therapeutic pad may then be provided to the user for application to a wound when and where desired.

The therapeutic pad is preferably attached to user's body at the wound site with a suitable adhesive which may be integral with the therapeutic pad or provided separately at the time of use. The therapeutic pad remains attached to the wound site for a period of time to allow the oxygen to contact and transfer to the wound site. After treatment, the therapeutic pad may be removed and replaced as necessary.

The therapeutic solution is prepared at least partially within the ultrasound station. A fluid is mixed with a gas containing oxygen within the ultrasound station. Other components may be added to achieve desired effects as described to achieve skin conditioning, skin rejuvenation and/or healing of wounded skin tissue.

The ultrasound station contains an ultrasound generator providing an electrical signal to an ultrasound transducer. The ultrasound transducer is induced to vibrate at an ultrasonic frequency to drive an ultrasound horn that is mechanically attached to the ultrasound transducer. The ultrasound vibrations travel down the ultrasound horn which includes an internal chamber. A fluid and a gas are introduced to the internal chamber which is subject to the ultrasound vibrations, providing intensive mixing between the fluid and the gas. Operating the internal chamber under pressure and using a gas with relatively pure oxygen, increases the amount of oxygen that is transferred to the therapeutic solution.

In its preferred embodiment, the therapeutic pad outer surface consists of an occlusive covering or impermeable layer over the matrix. This is useful in preventing loss of the oxygen to the atmosphere, extending the life of the therapeutic pad. Furthermore, occlusive coverings that maintain a moist environment may promote wound healing.

Although an occlusive layer can reduce oxygen brought from the exterior, a hypoxic condition in the present invention is avoided by the presence of the added oxygen. This may encourage angiogenesis, collagen synthesis and epithelialization. Moreover, various clostridium species, e.g., C. perfringens and C. septicum, are induced to germinate under hypoxic conditions, which can also support other anaerobic flora. In addition to minimizing anaerobic flora by discouraging germination, hyperoxic conditions are known to reduce the concentration of other pathogens as well.

Often the therapeutic pad will include a pressure sensitive adhesive forming an adhesive layer around the outer circumference of the inner surface to form a seal with the skin around the wound site.

Prior to storage of the therapeutic pad, a release liner may be applied to the adhesive layer to protect the adhesive layer during until use. The release line is removable from the adhesive layer at the time of use.

In another embodiment, a permeation layer may be placed between the matrix and the bottom surface of the therapeutic pad to control the release of materials such as oxygen from the matrix to the wound site.

A fluid adsorption layer may be placed between the occlusive layer and the adhesive layer to adsorb excessive drainage from the wound.

The present invention is an apparatus that is capable of providing one or more gases to a target area. One embodiment of the invention is a multi-layer wound dressing comes pre-filled with high levels of oxygen between the layers. The top layer is a barrier film that holds the oxygen over the wound, while the bottom layer is a high transfer rate film, attached over the wound. This self-contained dressing is applied to the wound like conventional wound dressings, and can be manufactured with a similar size, weight and feel of conventional dressings or transdermal patches.

The impermeable layer holds the oxygen in the vicinity of the wound, while the permeable or porous layer allows the oxygen to diffuse into the wound fluid at a rate proportional the gradient, until the wound fluid is saturated. The matrix acts like an oxygen reservoir, and as oxygen is consumed by the wound, there is a local abundant supply to be used as needed.

The therapeutic pad will accelerate healing of acute and chronic wounds, as well as provide skin conditioning and rejuvenation in addition to antibacterial and antifungal benefits.

In preferred embodiments of the present invention, methods and compositions are provided that comprise a material and a process for making a matrix that contains an entrapped gas, preferably gaseous oxygen. The matrix may comprise a natural or synthetic polymer that forms a closed-cell foam structure. Preferably, the cells of the foam are highly enriched for gaseous oxygen and the walls of the foam cells are enriched for dissolved oxygen. This material is useful as a primary tissue contact matrix where it is desirable to transfer oxygen into the tissue environment to increase the oxygen tension. A preferred embodiment is a polyacrylate matrix that is also flexible, elastic, conformable and highly absorbent comprising an optimal wound dressing matrix.

Other substrates comprising formations of closed-cell foams for the delivery of oxygen to tissues are contemplated by the present invention. For example, natural polymers of gelatin, dextrose, collagen, agar and agarose possess necessary molecular architecture for the encasement of gases such as oxygen within closed-cells to form a foam-like structure.

Similarly other water swellable cross-linked polymers such as polyacrylate, polymethacrylamide, polyester, polyether and polyurethane can entrap gases such as oxygen in close cell reservoirs within the matrix for delivery to compromised tissues. Furthermore, certain water non-swellable polymers such as silastic and silicone elastomer polymers may entrap gases such as oxygen within closed-cell structures.

The methods, compositions and devices of the present invention may be used to simultaneously deliver at least one active agent to a site. Agents such as moisturizers, cosmetics, rejuvenates, antimicrobial agents, antifungal agents, antiviral agents, growth factors, angiogenic factors, anaesthetics, mucopolysaccharides and other proteins may be incorporated into the compositions and devices for release into the environment. Especially preferred compositions and devices comprise a matrix that delivers both oxygen and another active agent that has enhanced activity because of the presence of the oxygen. For example, certain therapeutic agents are relatively inactive under reducing conditions but become significantly more active when conditions become more oxygenated. Adjuvants and other agents, such as those that boost the immune system, may also be incorporated into the devices of the present invention. An advantage of having agents directly incorporated into micro-cavities of the matrix is that the activities of those agents not altered by incorporation into the devices may be more effective upon their release.

Accordingly, it is an object of the present invention to provide compositions and methods for the delivery of oxygen.

Another object of the present invention is to provide compositions, methods and devices for the treatment of compromised tissue.

A further object of the present invention is to provide compositions, methods and devices comprising materials that enable the management of oxygen tension in a localized environment.

Yet another object of the present invention is to provide compositions, methods and devices comprising incorporation of active agents.

Still a further object of the present invention is to prevent infection by providing compositions, methods and devices that provide oxygen to anerobic sites.

In yet another object of the present invention, compositions, methods and devices are provided that deliver active agents, with or without the delivery of oxygen, to compromised tissue sites, for the prevention of infection and to aid in healing.

Another object of the present invention is to provide compositions, methods and devices that deliver oxygen for the enhancement of the activity of active or therapeutic agents.

It is another object of the present invention to provide compositions, methods and devices that easily conform to the shape of a compromised tissue site.

It is yet another object of the present invention to provide compositions and devices that are easily manufactured.

Still another object of the present invention is to provide compositions, methods and devices that may be easily removed from compromised tissues and replaced.

Yet another object of the present invention is to provide compositions, methods and devices that provide skin conditioning and skin rejuvenation therapy.

It is yet another object of the present invention to provide compositions, methods and devices that function to both absorb wound exudate and promote autolytic debridement.

Another object of the present invention is to provide compositions and methods for making single unit construction devices having multiple strands.

It is another object of the present invention to provide methods and compositions for treating compromised tissues using devices that function to both absorb moisture, deliver oxygen and deliver active agents.

An object of the present invention to provide methods and compositions for treating wounds using wound dressing devices having active agents incorporated therein.

Still another object of the present invention is to provide methods and compositions for delivering active agents to wound sites and damaged tissue.

A further object of the present invention is to provide tissue contact material that entraps gaseous oxygen or other gases to form a closed-cell foam.

### BRIEF DESCRIPTION OF THE DRAWINGS

The present invention will be shown and described with reference to the drawings of preferred embodiments and clearly understood in details.

FIG. 1 is a schematic representation of an embodiment of the ultrasound station in use.

FIG. 2 is a cross-sectional view of the therapeutic pad within a storage container.

FIG. 3 is a cross-sectional view of an embodiment of the therapeutic pad and other components within a storage container.

FIG. 4 is a cross-sectional view of an embodiment of the therapeutic pad covering a wound site.

FIG. 5 is a representation of an embodiment of the invention showing the therapeutic solution applied to a gel type matrix.

FIG. 6 is a representation of an embodiment of the invention showing the therapeutic solution applied to a gauze type matrix.

FIG. 7 depicts a flow chart illustrating a sequential embodiment of the method of treating wounds utilizing ultrasonic vibrations to create a therapeutic solution for assembly into a therapeutic pad for portable topical hyperbaric wound treatment.

FIG. 8 is a cross-sectional view of an embodiment of an ultrasound horn.

FIG. 9 is a cross-sectional view of an alternative embodiment of an ultrasound horn.

FIG. 10 is a cross-sectional view of an alternative embodiment of an ultrasound horn.

FIG. 11 is a cross-sectional view of an alternative embodiment of an ultrasound horn.

FIG. 12 is a cross-sectional view of an alternative embodiment of an ultrasound horn.

FIG. 13 is a cross-sectional view of an embodiment of an ultrasound horn distal end including the radiation surface.

FIG. 14 is a cross-sectional view of an alternative embodiment of an ultrasound horn distal end including the radiation surface.

FIG. 15 is a side elevation view of an alternative embodiment of an ultrasound horn distal end including the radiation surface.

FIG. 16 is a cross-sectional view of an alternative embodiment of an ultrasound horn distal end including the radiation surface.

FIG. 17 is a cross-sectional view of an alternative embodiment of an ultrasound horn distal end including the radiation surface.

### DETAILED DESCRIPTION OF THE DRAWINGS

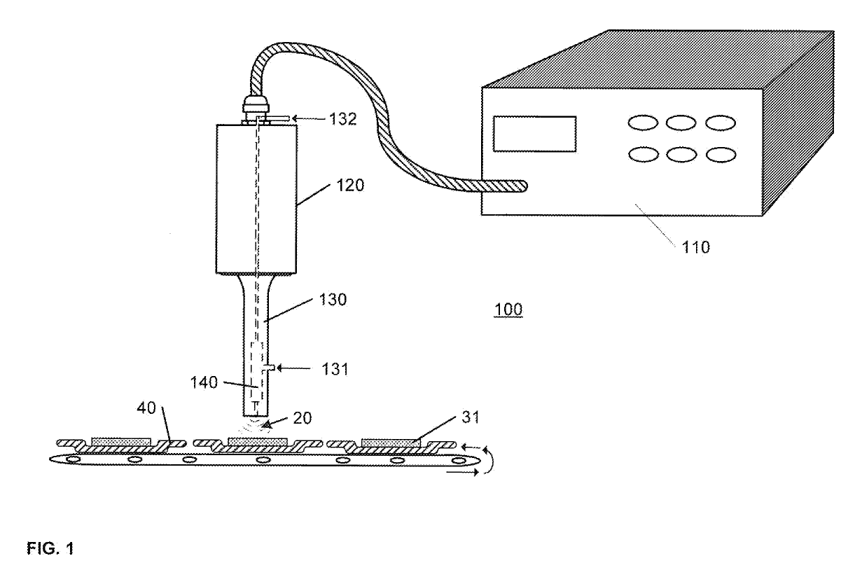

In reference to FIGS. 1-17, the present invention is directed towards apparatus and methods for promotion of skin healing on and tissue repair. More specifically, the invention is directed toward the manufacture of a storable therapeutic pad 30 containing a therapeutic solution 20 within a matrix 31 for use in wound healing, skin rejuvenation and skin conditioning. The therapeutic solution 20 is produced by mixing a gas 22 such as oxygen with a fluid 21 such as normal saline using an ultrasound station 100 to produce concentrations of oxygen far exceeding the normal amount of oxygen available under standard atmospheric conditions. The therapeutic pad 30 is attached to skin to provide the desired therapeutic benefit.

FIG. 1 is a schematic representation of an embodiment of the ultrasound station in use. The ultrasound station 100 includes an ultrasound generator 110 providing an electrical driving signal to generate mechanical vibrations within an ultrasound transducer 120. The mechanical vibrations would then be transmitted through an ultrasound horn 130. The ultrasound horn 130 proximal end is attached to the ultrasound transducer 120. The ultrasound horn contains an internal chamber 140 for mixing a therapeutic solution 20 which is discharged from the ultrasound horn 130 distal end to either itself form a matrix 31 or be applied to other materials to be incorporated into a matrix 31. Preferably oxygen is supplied to the internal chamber 140 through a radial supply channel 131 and normal saline is supplied to the internal chamber 140 through an axial supply channel 132. The matrix 31 may be located in an open portion of the storage container 40 as the therapeutic solution 20 is applied.

Preferably, to obtain optimal results, manufacturing would be completed in processes using controlled atmospheric packaging to maintain the product integrity. These processes include packaging with high barrier properties that contain the desired ratio of gases to preserve the product. Manufacturing and packaging under conditions of a substantially pure oxygen environment and elevated pressures ranging from about atmospheric to up to 100 psi, beneficially enhance the adsorption and oxygen carrying capacity of the matrix 31 while still maintaining conditions in which products may be safely manufactured and stored for later use.

Typically, maximum ambient oxygen concentrations of approximately 10 mg/l may be obtained in normal saline solutions. Under conditions of this invention, oxygen concentrations above 50 mg/l may be obtained in normal saline solution. Concentrations as high as 200 mg/l can be obtained under certain conditions at the higher pressure levels by supersaturating the matrix 31 as described.

Since oxygen is introduced into solution at relatively low pressures in aeration processes, the oxygen bubbles are relatively large, for example approximately 1 millimeter diameter. As a result, the aggregate bubble surface area for a dispersion of bubbles produced by bubble aeration is relatively small. The limited surface area produced by bubble aeration limits the concentration of gas that can be dissolved into solution. Oxygen dissolution is a function of the interfacial contact area between gas bubbles and the surrounding medium, and bulk fluid transport (mixing) in the liquid phase. In particular, the rate of oxygen dissolution is directly proportional to the surface area of the bubbles. A dispersion of very small bubbles, e.g. bubbles having diameters in the order of less than 50 microns, will have a much larger total surface area than a dispersion of large bubbles occupying the same volume. In the present invention, bubble diameters as low as 2 microns may be produced under certain conditions. In conventional processes the rate of oxygen dissolution in bubbling aeration is limited by the size of the bubbles introduced into the solvent. Fluid mixing is also very limited in conventional aeration because the only energy source available for agitation is the isothermal expansion energy of oxygen as it rises in the solution.

In the present invention, ultrasound energy is used to mix with a vigorous intensity the materials within the internal chamber 140. The ultrasonic waves may be generated having a frequency between 15 kHz and 40 MHz with a preferred frequency range of approximately 20 kHz to approximately 40 kHz. The recommended low-frequency ultrasound value is approximately 30 kHz and the recommended high-frequency ultrasound value is approximately 3 MHz.

The amplitude of the ultrasound waves can be 1 micron and above. The preferred amplitude range for low-frequency ultrasound is approximately 40 microns to approximately 60 microns, and the recommended amplitude value for low-frequency ultrasound is approximately 50 microns.

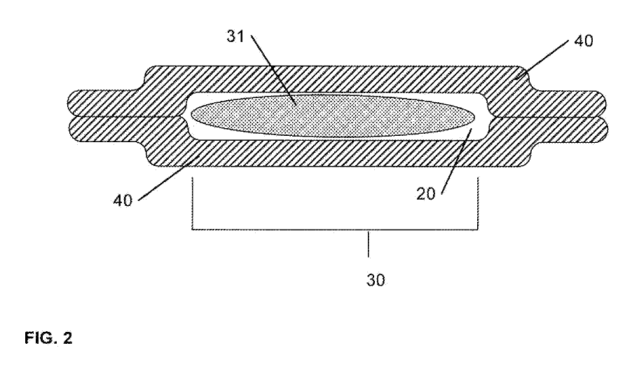

As shown in FIG. 2, the therapeutic pad 30 is stored within a storage container 40 until use. The storage container 40 may also include additional therapeutic solution 20 to supplement what is stored on or within the matrix 31.

Using packaging techniques known in the art, the storage container 40 is selected to be sealable and relatively non-permeable to gases and/or moisture. Potential embodiments of a storage container 40 include a blister-pack, can, jar, carton or similar package. The storage container 40 is designed and constructed to withstand design pressures of at least atmospheric pressures. Preferably the storage container 40 will have a burst strength of at least approximately 100 pounds per square inch (psi) to provide a safety factor for routine use with initial filling pressures possibly in the 30 to 50 psi range. Typically the storage container 40 will be of a single use design. Preferably the storage container 40 may be readily opened without the use of tools. Examples of this feature include a pull-tab, pull-string, perforated release point or tear tab feature in the storage container 40. By way of example, the storage container may be constructed of metal, plastic, foil-lined cardboard or combinations of materials.

The matrix 31 is configured to store a therapeutic solution 20 with any associated oxygen bubbles as a component of the therapeutic pad 30 which is applied to a patient's wound site. The matrix 31 is typically of a liquid, gel, solid or fibrous structure or various combinations of these. Examples of a liquid based matrix 31 include normal saline, alcohol, glycerin or other liquids compatible with wound treatment.

A gel or solid type matrix 31 may be placed to receive the therapeutic solution 20 from the ultrasound horn 130 or it may be formed from the therapeutic solution 20 itself. For example, polymers may be mixed with oxygen and the cross-linking agent in the ultrasound horn 130 and allowed to cure or set after sonication in the ultrasound horn 130.

Examples of natural polymers that may be used as a matrix 31 include; ethyl cellulose, cellulose derivatives, zein, gelatin, shellac, waxes, gums, natural rubber and chitosan.

Examples of synthetic elastomers that may be used as a matrix 31 include; polybutadiene, hydrin rubber, polyisobutylene, silicon rubber, nitrile, acrylonitrile, neoprene and butylrubber.

Examples of synthetic polymers that may be used as a matrix 31 include; polyvinyl alcohol, polyvinylchloride, polyethylene, polypropylene, polyacrylate, polyamide, polyurea, polyvinylpyrrolidone and polymethylmethacrylate.

A preferred composition of the present invention is a matrix 31 comprising a polymer, a non-gellable polysaccharide, and one or more active agents incorporated therein. For example, a matrix 31 may contain an acrylamide polymer, guar gum, and one or more active agents incorporated therein.

The compositions and devices of the present invention may take many physical forms, depending on uses of the compositions and devices. A preferred shape is a gel sheet that can be cut or molded into any two dimensional shape. Other preferred embodiments are primarily constructed of thin strands of matrix 31 suitable for placement into the wound bed or cavity. The preferred devices may be constructed from one or multiple strands of matrix. When multiple strands are used in the construction, the strands are secured together by wrap, tie, glue, or alternatively by a continuous bridge of matrix between adjacent strands. Multiple strands are secured together to minimize accidental loss during removal of the dressing from the wound bed.

The fibrous or stranded embodiment enables the device to maintain its integrity and also maximize the surface area to volume ratio of its matrix 31. This is important since the matrix 31 may be an absorbent material where a high surface area to volume ratio increases the rate of absorption, without increasing the overall absorption capacity of the device.

In addition, the stranded matrix construction maximizes the overall flexibility and pliability of the dressing. In embodiments of the device where multiple strands are employed, the overall flexibility and conformational characteristics of the device are maintained by binding strands in only limited and restricted areas.

Another advantage of the stranded matrix construction is the “semi-porous” quality of the wound dressing that allows for the removal of extraneous cellular matter resulting during the wound healing process. The air in the inter-strands area of the device serve as a reservoir of space that may be displaced allowing for the removal of excess materials such as exudate fluid, debridement product and cellular exudate from the wound bed.

Therapeutic pads 30 of the present invention may be produced by cutting a desired design pattern from stock sheets of matrix 31 material. For example, the matrix 31 may be die-cut from stock sheets of an absorbent polyacrylate wound dressing material. The stranded cut-out may then be coated with an agent to prevent aggregation and tangling of the free floating strands. Coating agents that may be used include, but are not limited to, petrolatum, talcum, polyglycols, glycerol, propylene, glycol, vegetable oil, and animal oil. Following the steps of cutting and coating, the material may be sterilized using sterilization techniques known in the art such as gamma radiation, steam and heat sterilization, electron beam or chemical sterilization (such as by use of ethylene oxide).

In use, the therapeutic pad 30 of the present invention are the primary dressing placed in direct contact with the wound bed, or as near as practical against the wound bed. The devices may serve as a packing material and, if required, may be secured into position with any suitable secondary wound dressing such as a wrap, tape, gauze, or pad. The dressings are temporary, however, and are not intended for permanent incorporation into the healed tissues. When necessary, the therapeutic pad 30 is changed by first removing any over-dressing material and then removing the device, whereby any accumulated necrotic tissue and exudate is lifted away. The therapeutic pad 30 of the present invention may be replaced by a fresh device or other suitable wound covering.

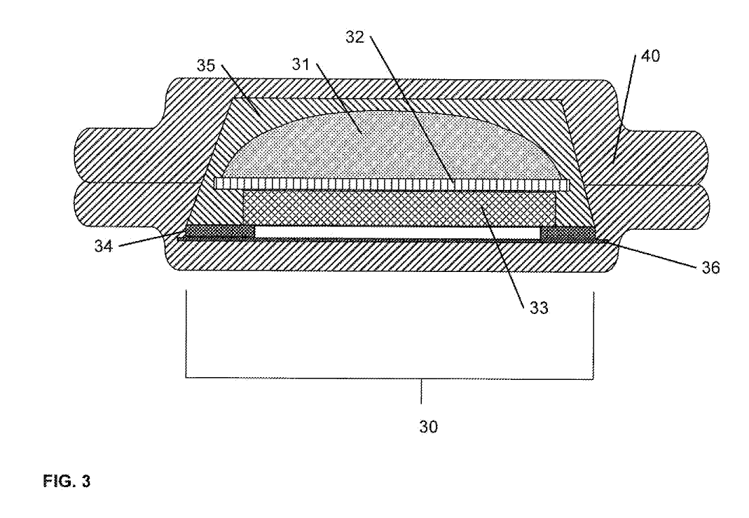

As shown in FIG. 3, the therapeutic pad 30 in addition to the matrix 31 may include a permeation layer 32 to control the transfer of oxygen and/or other materials, a fluid adsorption layer 33 to adsorb excess fluid secretions from the wound area, an adhesive layer 34 to attach the therapeutic pad 30 to the patient's skin tissue, an impermeable layer 35 to reduce loss of oxygen to the atmosphere and a release liner 36 to protect the adhesive layer 34 while in storage from the time between manufacture and application to the patient's skin. These components, when used, may be preassembled during manufacture and stored within the storage container 40 as shown in FIG. 3.

A fluid adsorption layer 33 may be located as desired between the impermeable layer 35 and the adhesive layer 34. The fluid adsorption layer 33 may be manufactured from cellulosic materials, polyvinyl alcohol-acetal or hydrogel, for example. In one embodiment, the fluid adsorption layer 33 is composed of an absorbent synthetic polyacrylate material. The rate of absorption of polyacrylate is significantly increased by cutting the material into strands, which increases the surface area to volume ratio. This also provides a greater surface area for the release of dissolved oxygen and other active agents from the device. Polyacrylate material is particularly suitable because it retains its integrity during interaction with wound exudate moisture, as well as with necrotic tissue and wound debris. The wound dressing device of the present invention does not dissolve, gel or otherwise disintegrate during application to the wound. The preferred matrix swells slightly during the absorption of moisture, causing the device to conform closely to the walls of the wound bed.

An adhesive layer 34 is the material that helps in maintaining an intimate contact between the therapeutic pad 30 and the skin surface. It should adhere with not more than applied finger pressure, be aggressively and permanently tacky, and exert a strong holding force. Additionally, it should be removable from the smooth surface without leaving a residue 37-38. Polyacrylates, polyisobutylene polysiloxanes, polyacrylates, polyisobutylene and silicon based adhesives are widely used as adhesive layers 34. The selection of an adhesive is based on numerous factors, including the pad design and drug formulation. The adhesive layer may serve multiple functions such as a permeation layer 32 or a fluid adsorption layer 33.

During storage the therapeutic pad 30 is covered by a release liner 36 which is a protective liner that is removed immediately before the application of the therapeutic pad 30 to the skin. It is therefore regarded as a part of the primary packaging material rather than a part of dosage form for delivering the drug. Typically, a release liner 36 is composed of a base layer which may be non-occlusive (e.g. paper fabric) or occlusive (e.g. polyethylene, polyvinylchloride) and a release coating layer made up of silicon or teflon. Other materials used for release liners 36 include polyester foil and metallized laminates.

An impermeable layer 35 designed to contain oxygen and moisture within the therapeutic pad 30 during use. The impermeable layer 35 preferably does not irritate the skin during long wear. The most comfortable backing will be the one that exhibits lowest modulus or high flexibility. Examples of some backing materials are vinyl, polyethylene and polyester films which may be constructed of metallized polyester, ceramic coated polyester, polyvinylidene chloride laminates such as Saranex®, ethylene vinyl alcohol (EVA) laminates such as Oxyshield®, or polyamide laminates such as Capran®.

In one embodiment, the oxygen stored within the therapeutic pad 30 is controllably released to the user through the permeation layer 32. By varying the composition and thickness of the membrane, the dosage rate per unit area of the device can be controlled. For example, PVC, polyethylene, ethylene vinyl acetate, EVA, ethyl cellulose, silicon rubber and polyurethanes. The permeation layer 32 is a permeable film is configured to be permeable to gases. For example, the permeation layer 32 may be constructed of polyurethane, silicone, polyvinylchloride, polyolefins, and the like, preferably ethylene vinyl alcohol (EVA) or EVA/polyethylene.

The amount of oxygen released to the user while wearing the therapeutic pad 30 may vary according to the concentration of the gas contained within the matrix 31 and the material used as the permeation layer 32. Other factors such as temperature and atmospheric pressure may also affect the amount of oxygen released to the user.

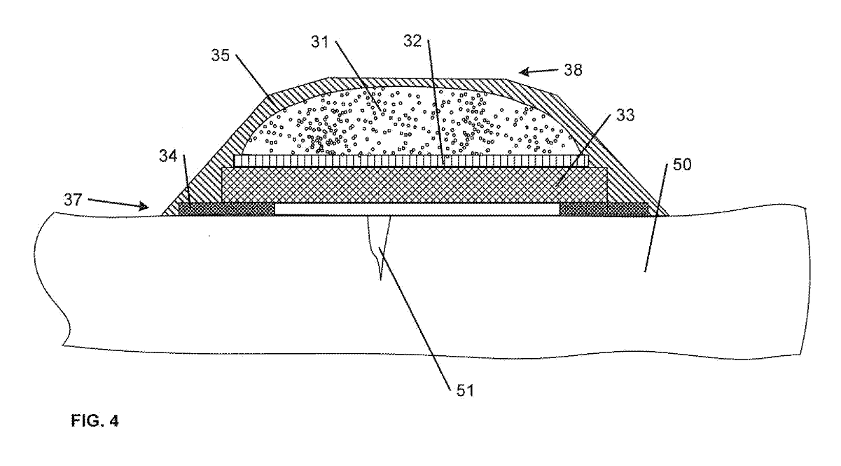

FIG. 4 is a cross-sectional view of an embodiment of the therapeutic pad covering a wound site. In this embodiment, the therapeutic pad 30 having an impermeable layer 35 exposed to the atmosphere forms an outer surface 38. The storage container 40 and the release liner 36 of the therapeutic pad 30 have been removed from the adhesive layer 34 exposing has an inner surface 37 of the therapeutic pad 30 that is attached to the skin 50 surrounding the wound site 51. Preferably the adhesive layer 34 adheres the therapeutic pad 30 to the skin 50. Further, the adhesive layer 34 may also be utilized to prevent the gas that is delivered through the permeation layer 32 to the user from escaping. In one embodiment, the adhesive layer 34 may cover the perimeter of the therapeutic pad 30. In another embodiment, the adhesive layer 34 may cover the entire therapeutic pad 30 and may be integrated with the permeation layer 32.

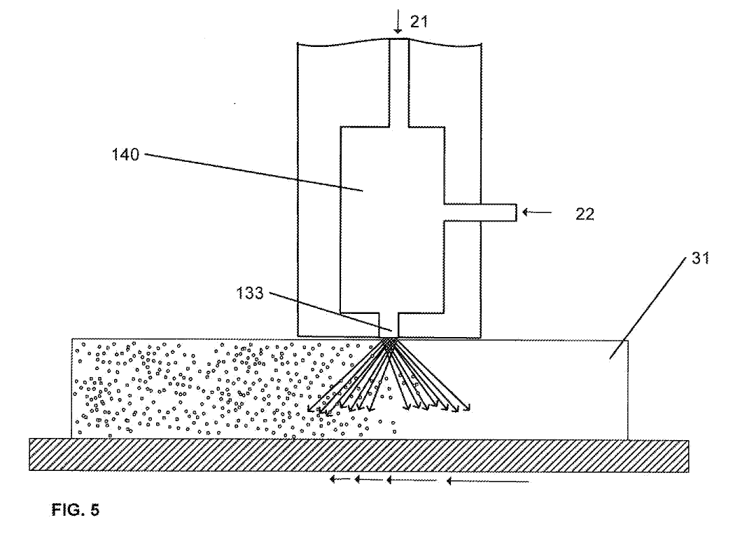

FIG. 5 is a representation of an embodiment of the invention showing the ultrasound waves showing the therapeutic solution 20 applied to a gel type matrix 31. The fluid 21 and gas 22 entering the internal chamber 140 are subject to intensive agitation producing an oxygenated mixture having a oxygen content well above the equilibrium limit at ambient conditions. The oxygenated mixture can supply a large amount of molecular oxygen in a medium that is not traumatic to skin tissue. Since the dissolution of oxygen into solution occurs under hyperbaric conditions, a large concentration of oxygen is dissolved into solution. The resulting therapeutic solution 20 can have a dissolved oxygen content as high as 200 mg/l.

In one embodiment of the therapeutic solution 20, an oxygen-enriched solution is accompanied by a dispersion of micro-bubbles through the discharge channel 133 into the matrix 31. In another embodiment, the oxygenated solution and micro-bubble dispersion are encapsulated in a matrix 31 with properties of a Bingham Plastic.

High shear forces and small bubble size allow the therapeutic solution containing micro-bubbles to disperse within a matrix 31. Gas micro-bubbles that may be dispersed or nucleate from solution, where the solution is a Newtonian fluid, such as water, rise to the surface and are released into the air above the solution. Gas bubbles rise in such fluids because a net body force exists that projects the bubbles upward. Since Newtonian fluids yield to these forces, the bubbles rise. These mechanics, which control bubble rise, are explained by Stokes Law. In some applications, it is desirable to limit or substantially prevent bubbles from rising to the surface of the solution during storage and to maintain the micro-bubble dispersion indefinitely. In particular, it may be commercially desirable to market a product that contains visible oxygen bubbles that are held indefinitely in a suspension.

A supersaturated solution of oxygen-matrix 31 system will attempt to reject oxygen by nucleating oxygen bubbles. Nucleation can be either a homogeneous or heterogeneous process, depending on changes in temperature, mechanical agitation, or the presence of suitable particles that can stimulate gas nucleation. Rapid pressure changes can provoke gas bubble nucleation, and in this invention, a reduction of pressure to ambient will typically result in the formation of micro-bubbles.

The micro-bubble therapeutic solution 20 is characterized as having a very large surface area through which interfacial transport of oxygen occurs. Interfacial transport of oxygen through a large surface area aids in resupplying oxygen to solution when dissolved oxygen is taken up during chemical reactions. As a result, a large surface area in the micro-bubble dispersion is desirable.

The matrix 31 preferably contains micro-bubbles having an average bubble diameter of about 1-100 microns. Micro-bubbles within this size range provide a significantly larger surface area than a cluster of large bubbles containing the same volume of gas.

One novel aspect of this invention involves the substitution of a Newtonian solvent such as normal saline with a Bingham Plastic. Such a material requires a finite yield stress to initiate movement. An important characteristic of a Bingham Plastic is that the yield stress. Applied stress levels that are below the yield stress threshold will not result in movement of the fluid. A Bingham Plastic can be considered to have infinite viscosity and behave as a solid at stress levels below the yield stress.

It can be seen from Stokes' Law, a Bingham Plastic will result in bubble immobilization, provided that the magnitude of the buoyancy forces exerts a stress level that falls below the yield stress for the Bingham Plastic type matrix 31. Bubble immobilization will provide stability of the micro-bubble suspension. The Bingham plastic type matrix 31 is characterized as having a finite yield stress. Fluid movement in a Bingham plastic type matrix 30 will not occur until the finite yield stress is exceeded. Once the yield stress has been exceeded, the stress may increase linearly with increasing shear rate. Buoyancy forces acting on the oxygen micro-bubbles are insufficient to overcome the finite yield stress in the Bingham Plastic type matrix 30. Therefore, the Bingham Plastic type matrix 30 immobilizes the micro-bubbles in the mixture for extended periods.

It has been discovered that the current invention can produce stable suspensions of micro-bubbles when a Bingham Plastic is used as the therapeutic solution 20. This is preferably accomplished by adding and mixing the ingredients to form a Bingham Plastic and an oxygenated liquid at elevated pressure, i.e.: prior to the formation of micro-bubbles. The ultrasound station 100 is used to apply the energy to maintain the therapeutic solution 20 well above its yield stress. Since the components are mixed prior to the solution being reduced to ambient pressure, micro-bubbles will not substantially form. Once the solution is reduced in pressure, micro-bubbles will form; however, these bubbles are immobilized by the previously formed Bingham Plastic now being within the matrix 31 and below its yield stress having exited the ultrasound horn 130.

A variety of Bingham Plastics provide a suitable therapeutic solution 20, including but not limited to formulations using clay based thickening agents, such as Optigel-SH 8 manufactured by Sud-Chemie, Inc., and formulations using polymeric based thickening agents, such as Carbopol® polymers manufactured by B.F. Goodrich Company.

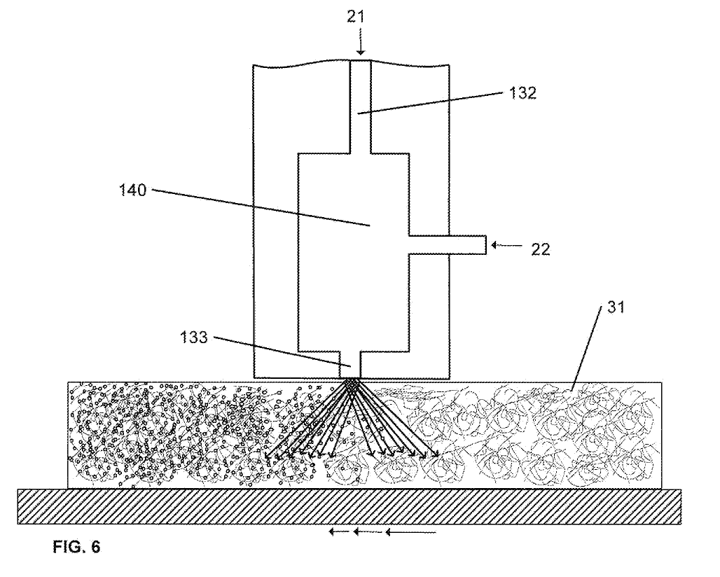

FIG. 6 is a representation of an embodiment of the invention showing the ultrasound waves showing the therapeutic solution 20 applied to a gauze type matrix 31.

Preferred embodiments of the present invention, particularly those used as wound dressing devices, may also take a particular conformation. For example, a preferred embodiment of the present invention comprises a stranded configuration wherein the individual strands extend from at least one common region and may have free floating ends. This particular conformation is particularly suitable for use in deep wounds since the multiple matrix strands enable the dressing to conform to individual and uniquely shaped wound areas. Furthermore, the devices accelerate wound healing by displacing and allowing for the removal of excess cellular exudate and debris, thereby improving the rate of tissue repair and regeneration. As described previously micro-bubbles may be formed in the therapeutic solution 20 within the ultrasound horn 130 as well as upon release of pressure upon opening the storage container 40. Micro-bubbles can become attached to solid surfaces through van der Waals forces, hydrogen bonding or electrostatic interactions. The gauze type matrix 31 provides a variety of surface areas upon which micro-bubbles may attach and be held in a relatively stable form until use.

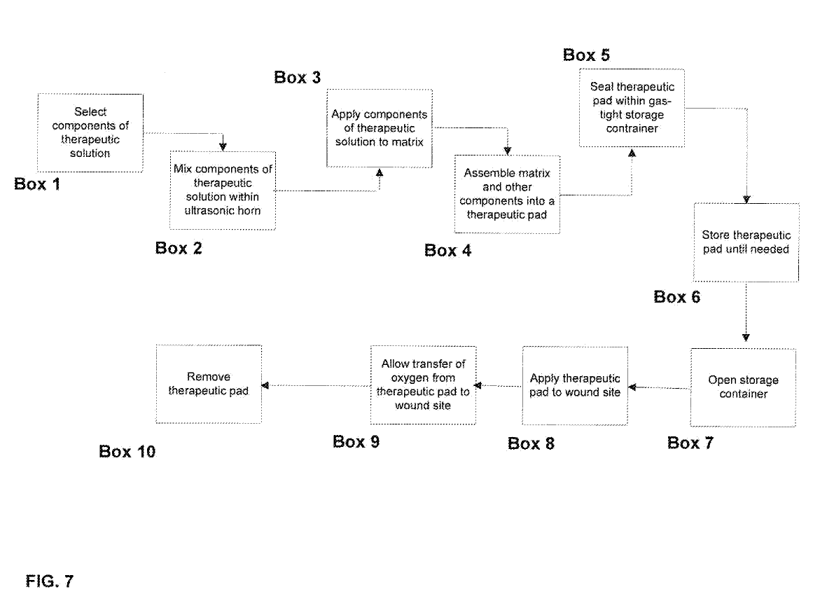

FIG. 7 depicts a flow chart illustrating a sequential embodiment of the method of treating wounds utilizing ultrasonic vibrations to create a therapeutic solution 20 for assembly into a therapeutic pad 30.

Box 1 represents the step of selecting components of therapeutic solution 20. Generally, these components may include liquids, solids and/or gases in various combinations as some of which have been described herein. Typically normal saline (0.9% sterile saline) will be combined with filtered oxygen gas to produce the therapeutic solution 20. Alternatively a gel may be used to capture oxygen micro-bubbles forming a multi-phased closed-cell foam.

In Box 2 the various components of therapeutic solution 20 are transferred to an internal chamber 140 within ultrasound horn 130 and subject to intensive ultrasound energy, preferably in resonance, at a frequency of approximately 16 kHz or greater, to produce the therapeutic solution 20.

As shown in Box 3, the therapeutic solution 20 may be applied to a matrix 31. Alternative embodiments in which the therapeutic solution 20 itself is used as the matrix 31 are also disclosed.

The matrix 31 and other components such as fluid adsorption layer 33, adhesive layer 34, impermeable layer 35 are then assembled into a therapeutic pad 30 as represented by Box 4.

In Box 5 the therapeutic pad 30 is sealed within gas-tight storage container 40 to preserve the quality of the therapeutic pad 30. The matrix 31 may contain oxygen levels at concentrations as high as 200 mg/l and suitable to withstand pressures from approximately atmospheric to up to 100 psi.

As represented by Box 6, the therapeutic pad 30 is distributed through conventional medical distribution marketing channels, provided to the appropriate medical personnel or user and stored until needed by a patient.

Box 7 represents breaking the seal of the storage container 40 to open the storage container 40 and remove the therapeutic pad 30. This of course releases any pressure gradient above ambient under which the therapeutic pad 30 may have been stored.

Box 8 represents applying the therapeutic pad 30 to the wound site 51. In the event, an adhesive layer 34 and/or an impermeable layer 35 were not included with the therapeutic pad 30 the therapeutic pad 30 can be covered and attached with conventional wound dressing materials of the appropriate size and properties to hold the therapeutic pad 30 in place.

Box 9 provides the step of use in which the oxygen or other therapeutic from therapeutic pad 30 is transferred to wound site and excess fluids may be taken up by the therapeutic pad 30 and stored in the fluid adsorption layer 34 when provided.

As the therapeutic pad 30 is a temporary use device, Box 10 represents removal of the therapeutic pad 30 and replacing it or providing other care to the skin as appropriate.

This method can be used to provide oxygen to anaerobic environments. In the presence of the matrix, anaerobic organisms will be killed, providing treatments for infections due to anaerobic organisms. One use for an oxygen-delivery devices such as the present invention, is in the control and elimination of strict anaerobic bacteria. Anaerobic bacteria have low or no tolerance for elemental oxygen and rapidly die if exposed to air or any other source of the gas. Pathogenic strains of these organisms tend to form localized anaerobic environments in tissues. The insertion of the present invention into such environments would serve to oxygenate the surrounding areas and thereby cause the death of the pathogens. Therefore, such a device has utility in the treatment of infectious gangrene.

Additionally, for skin rejuvenation, conditioning and/or wound care oxygen supplied can be used to activate active agents that are not very active without oxygen and thus, these agents can be used in anaerobic environments. One or more matrices can be used to provide both the oxygen and the agent activated by the oxygen to allow for treatments of tissues that are not normally treated in this manner. One use for a tissue contact material for the delivery of oxygen to compromised tissues is in adjunctive therapies that might be enhanced in activity by an elevation of the local oxygen tension. As an example, certain therapeutic agents are relatively inactive under reducing conditions but become significantly more active when conditions become more oxygenated.

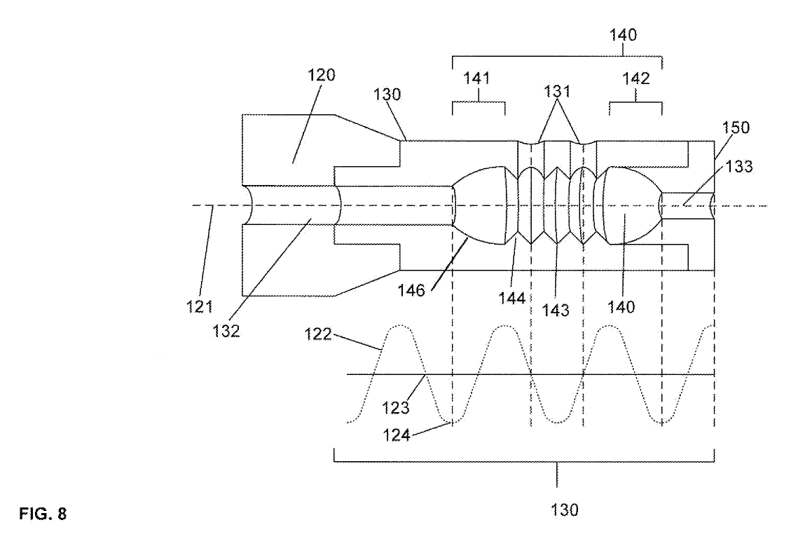

FIG. 8 illustrates an apparatus that may be utilized to create the therapeutic combination and/or spray it onto a wound to be treated. The apparatus comprises an ultrasound horn 130 and an ultrasound transducer 120 attached to the proximal surface of ultrasound horn 130 powered by generator 110. As ultrasound transducers and generators are well known in the art they need not and will not, for the sake of brevity, be described in detail herein. The ultrasound horns 130 utilized to create the therapeutic solution 20 and spray it onto the matrix 31 may be capable of vibrating in resonance at a frequency of approximately 16 kHz or greater. The ultrasonic vibrations traveling down the ultrasound horn 130 may have an amplitude of approximately 1 micron or greater. It is preferred that the ultrasound horn 130 utilized be capable of vibrating in resonance at a frequency between approximately 20 kHz and approximately 200 kHz. It is recommended that the ultrasound horn 130 be capable of vibrating in resonance at a frequency of approximately 30 kHz. The signal driving the ultrasound transducer may be a sinusoidal wave, square wave, triangular wave, trapezoidal wave, or any combination thereof.

Ultrasound horn 130 comprises a proximal surface, a radiation surface 150 opposite the proximal surface, and at least one radial surface extending between the proximal surface and the radiation surface 150.

Within the ultrasound horn 130 is an internal chamber 140 containing a back wall 141, a front wall 142, at least one side wall 143 extending between the back wall 141 and the front wall 142, and a rear ultrasonic lens 146 within back wall 141. As to induce vibrations within ultrasound horn 130, ultrasound transducer 120 may be mechanically coupled to the proximal surface. Mechanically coupling ultrasound horn 130 to ultrasound transducer 120 may be achieved by mechanically attaching (for example, securing with a threaded connection), adhesively attaching, and/or welding ultrasound horn 130 to ultrasound transducer 120. Other means of mechanically coupling ultrasound horn 130 and ultrasound transducer 120, readily recognizable to persons of ordinary skill in the art, may be used in combination with or in the alternative to the previously enumerated means. Alternatively, ultrasound horn 130 and ultrasound transducer 120 may be a single piece. When ultrasound transducer 120 is mechanically coupled to ultrasound horn 130, driving ultrasound transducer 120 with an lectrical signal supplied from ultrasound generator 110 induces ultrasonic waves 122 having at least one node 123 and one antinode 124 within the ultrasound horn 130. If ultrasound transducer 120 is a piezoelectric transducer, then the amplitude of the ultrasonic waves 122 traveling down the length of ultrasound horn 130 may be increased by increasing the voltage of the electrical signal driving the ultrasound transducer 120.

As the ultrasonic waves 122 travel down the length of the ultrasound horn 130, back wall 141 oscillates back-and-forth. The back-and-forth movement of back wall 141 induces the release ultrasonic vibrations from rear ultrasound lens 146 into the materials inside internal chamber 140. Positioning back wall 141 such that at least one point on rear ultrasound lens 146 lies approximately on an antinode 124 of the ultrasonic waves 122 passing through ultrasound horn 130 may maximize the amount and/or amplitude of the ultrasonic vibrations emitted into the materials in internal chamber 140. Preferably, the center of rear ultrasound lens 146 lies approximately on an antinode 124 of the ultrasonic waves 122. The ultrasonic vibrations emanating from rear ultrasound lens 146, travel towards the front of internal chamber 140. As to minimize the oscillations and/or vibrations of front wall 142, it may be desirable to position front wall 142 such that at least one point on front wall 142 lies on an antinode 124 of the ultrasonic waves 122. Preferably, the center of front wall 142 lies approximately on a antinode 124 of the ultrasonic waves 122.

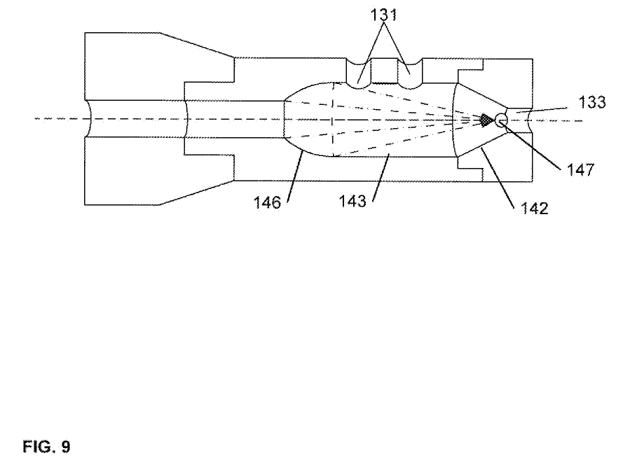

The specific lens illustrated in FIG. 9 contains a concave portion 123. If concave portion 123 forms an overall parabolic configuration in at least two dimensions, then the ultrasonic vibrations, depicted by the arrows, emanating from concave portion of rear ultrasound lens 146 travel in an undisturbed pattern of convergence towards the parabola's focus 147. As the ultrasonic vibrations 122 converge at focus 147, the ultrasonic energy carried by vibrations 122 may become focused at focus 147. The materials passing through internal chamber 140 are therefore exposed to the greatest concentration of ultrasonic energy at focus 147. Consequently, the ultrasonically induced mixing of the materials may be greatest at focus 147. Positioning focus 147 at or near the opening of discharge channel 133, as to be in close proximity to the opening of discharge channel 133 in front wall 142 may, therefore, yield the maximum mixing of the materials as the materials enter discharge channel 133.

The materials to be atomized and/or mixed enter internal chamber 140 of the embodiment depicted in FIG. 8 through at least one radial supply channel 131 originating in a radial surface and opening into internal chamber 140. Preferably, radial supply channel 131 encompasses a node 123 of the ultrasonic waves 122 traveling down the length of the ultrasound horn 130 and/or emanating from rear ultrasound lens 146. In the alternative or in combination, radial supply channel 131 may originate in radial surface 118 and open at back wall 141 into internal chamber 140. Upon exiting radial supply channel 131, the materials pass through internal chamber 140. The combined materials then exit internal chamber 140 through discharge channel 133, originating within front wall 142 and terminating within radiation surface 150. If the combination is primarily a fluid, the pressure of the combination decreases while its velocity increases as it passes through discharge channel 133. Thus, as the combination flows through discharge channel 133, the pressure acting on the combination may be converted to kinetic energy. If the combinations gains sufficient kinetic energy as it passes through discharge channel 133, then the attractive forces between the molecules of the combination may be broken, causing the combination to atomize as it exits discharge channel 133 at radiation surface 150.

It is preferable if at least one point on radiation surface 150 lies approximately on an antinode of the ultrasonic waves 122 passing through ultrasound horn 130.

As to simplify manufacturing, ultrasound horn 130 may further comprise an interchangeable radiation surface 150 attached to its distal end. Radiation surface 150 may be mechanically attached (for example, secured with a threaded connector), adhesively attached, and/or welded to the distal end of ultrasound horn 130.

FIG. 8 illustrates an alternative ultrasound horn 130 that may be used to create the therapeutic combination and/or spray it onto a wound characterized by at least one protrusion 144 along the side wall 143 and extending into the internal chamber 140. The incorporation of protrusions 144 may enhance ultrasonic echoing within internal chamber 140 by increasing the amount of ultrasonic vibrations emitted into internal chamber 140 and/or by providing a larger surface area from which ultrasonic vibrations echo.

The distal, or front facing, edges of protrusions 144 may emit ultrasonic waves into the chamber when ultrasound horn 130 is vibrated. The proximal, or rear facing, and front facing edges of protrusions 144 reflect ultrasonic waves striking the protrusions 144. Emitting and/or reflecting ultrasonic vibrations into internal chamber 140, protrusions 144 increase the complexity of the echoing pattern of the ultrasonic vibrations within internal chamber 140. The specific protrusions 144 depicted in FIG. 8 comprise a triangular shape and encircle the cavity. The protrusions may be formed in a variety of shapes such as, but not limited to, convex, spherical, triangular, rectangular, polygonal, and/or any combination thereof. In the alternative or in combination to being a band encircling the chamber, the protrusion may spiral down the chamber similar to the threading within a nut. In combination or in the alternative, the protrusions may be discrete elements secured to a side wall of chamber that do not encircle the chamber. In the alternative or in combination, the protrusions may be integral with side wall or walls of the chamber. Furthermore, protrusions 144 may be utilized to increase mixing within chambers containing convex and/or concave ultrasonic lenses within their front and/or back walls. In the alternative or in combination, protrusions 144 may be utilized to increase mixing within chambers lacking ultrasonic lenses within their front and/or back walls.

Alternative embodiments of an ultrasound horn 130 that may be utilized to create the therapeutic combination and/or spray it onto the body may possess a radial supply channel 131 opening within side wall 143 of internal chamber 140. FIG. 9 is a cross-sectional view of an alternative embodiment of an ultrasound horn having a concave front wall having a focus and a conical back wall. If multiple channels are utilized, they may also be aligned along the central axis 121 of ultrasound horn 130, as depicted in FIG. 9. Alternatively or in combination, radial supply channel 131 may be located on different platans, as depicted in FIG. 9, and/or at different radial positions around the same platan.

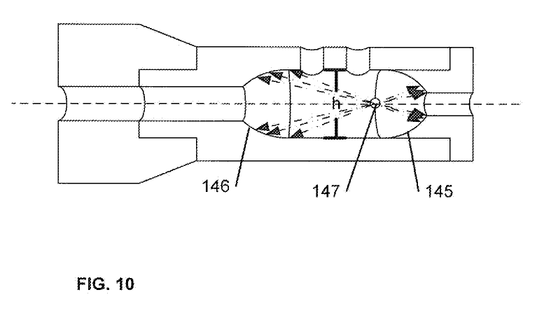

FIG. 10 is a cross-sectional view of an alternative embodiment of an ultrasound horn having a concave front wall and a concave back wall. In this embodiment the parabolas formed by concave portions of front ultrasound lens 145 and rear ultrasound lens 146 have a common focus 147. In the alternative, the parabolas may have different foci. However, by sharing a common focus 147, the ultrasonic vibrations emanating and/or echoing off the parabolas and/or the energy the vibrations carry may become focused at focus 147. The materials passing through internal chamber 140 are therefore exposed to the greatest concentration of the ultrasonic agitation, cavitation, and/or energy at focus 147. Consequently, the ultrasonic induced mixing of the materials is greatest at and/or about focus 147. Positioning focus 147, or any other focus of a parabola formed by the concave portions, at point downstream of the entry of at least two materials into internal chamber 140 may maximize the mixing of the fluids entering internal chamber 140 upstream of the focus.

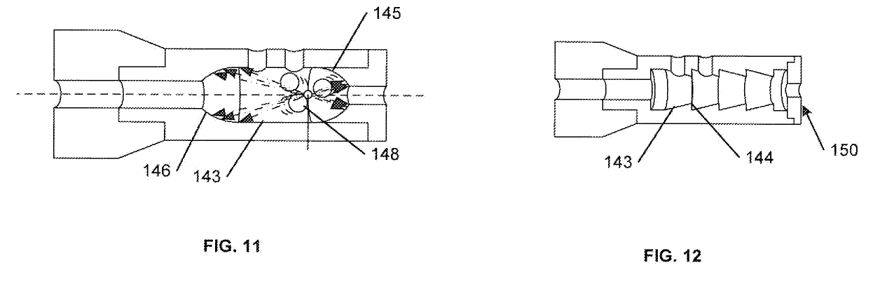

FIG. 11 is a cross-sectional view of an alternative embodiment of an ultrasound horn having a free member 148 within internal chamber 140. Ultrasonic vibrations emanating from rear ultrasound lens 146 within back wall 141 and/or echoing of front ultrasound lens 145 within front wall 142 may induce free members 148 to move about internal chamber 140. Traveling through internal chamber 140, ultrasonic vibrations strike free members 148 and push them in the direction of ultrasonic waves 122. As free members 148 move about internal chamber 140 they mechanically agitate the materials within chamber causing the materials to mix.

FIG. 12 is a cross-sectional view of an alternative embodiment of an ultrasound horn 130 that may be used to create the therapeutic combination and/or spray it onto a wound characterized by at least one protrusion 144 on the side wall 143 and extending into internal chamber 140 comprising a back facing edge and a front facing edge less streamlined than the back facing edge. As with the embodiment depicted in FIG. 8, the incorporation of protrusions 144 may enhance ultrasonic echoing within internal chamber 140 by increasing the amount of ultrasonic vibrations emitted into internal chamber 140 and/or by providing a larger surface area from which ultrasonic vibrations echo. In combination or in the alternative, protrusions 144 may generate a pumping action when ultrasound horn 130 is vibrated in resonance. As previously stated, vibrating ultrasound horn 130 in resonance induces segments of the horn to expand and contract as ultrasonic waves 122 travel down the length of the horn. As ultrasound horn 130 expands, the less streamlined front facing edges move forward. As the front facing edges move forward, they push the materials within internal chamber 140 towards discharge channel 133. Likewise, when the horn contracts, the more streamlined rear facing edges push the material away from discharge channel 133. However, because the rear facing edges are more streamlined then edges 602, more fluid is pushed forwards then backwards. Consequently, an overall forward pumping action is produced by the expansion and contraction of protrusions 144.

Regardless of the specific horn utilized, ultrasonic vibrations emanating from the horn's radiation surface may atomize the combination exiting the horn into a spray. The ultrasonic vibrations may also direct and/or confine the spray. The manner in which ultrasonic vibrations emanating from the radiation surface 150 direct the spray ejected from the horn utilized depends largely upon the conformation of radiation surface 150

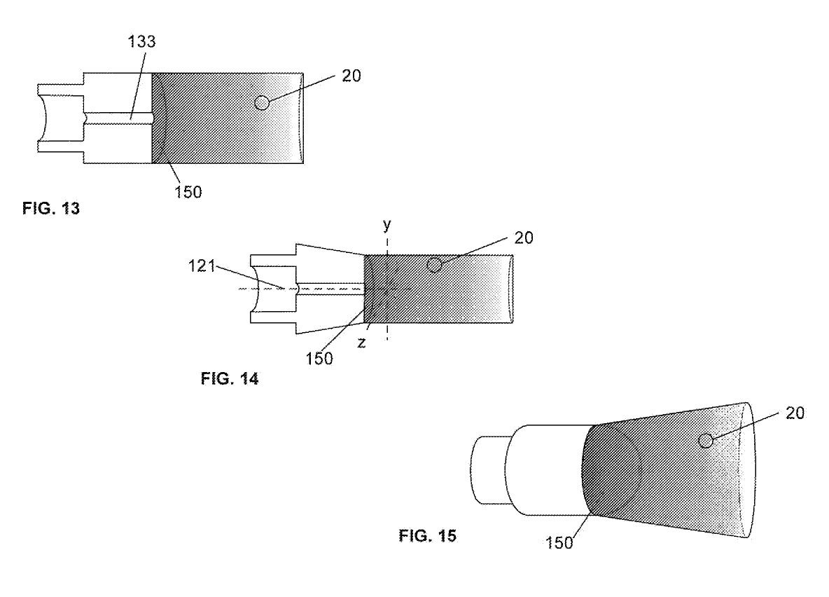

FIGS. 13 and 14 depict radiation surfaces 150 comprising a planar face producing a roughly column-like spray pattern. Radiation surface 150 may be tapered such that it is narrower than the width of the horn in at least one dimension oriented orthogonal to the central axis 121 of the horn, as depicted FIG. 14. Ultrasonic vibrations emanating from the radiation surface 150 depicted in FIGS. 13 and 14 may direct and confine the vast majority of spray ejected from discharge channel 133 to the outer boundaries of the radiation surfaces 150. Consequently, the majority of spray emitted from channel 133 in FIGS. 13 and 14 is initially confined to the geometric boundaries of the respective radiation surfaces.

Regardless of the configuration of the radiation surface, adjusting the amplitude of the ultrasonic vibrations traveling down the length of the horn utilized may be useful in focusing the spray exiting the horn. The amount of focusing obtained by the ultrasonic vibrations emanating from the radiation surface and/or the ultrasonic energy the vibrations carry depends upon the amplitude of the ultrasonic vibrations traveling down horn. As such, increasing the amplitude of the ultrasonic vibrations may narrow the width of the spray pattern produced; thereby focusing the spray produced. For instance, if the spray exceeds the geometric bounds of the radiation surface, i.e. is fanning too wide, increasing the amplitude of the ultrasonic vibrations may narrow the spray. Conversely, if the spray is too narrow, then decreasing the amplitude of the ultrasonic vibrations may widen the spray. If the horn is vibrated in resonance frequency by a piezoelectric transducer attached to its proximal end, increasing the amplitude of the ultrasonic vibrations traveling down the length of the horn may be accomplished by increasing the voltage of the electrical signal driving the transducer.

FIG. 15 is an elevational view of an alternative embodiment of an ultrasound horn distal end including the radiation surface 150. The ultrasonic vibrations emitted from the convex portion of the radiation surface 150 depicted in FIG. 15 directs spray radially and longitudinally away from radiation surface 150.

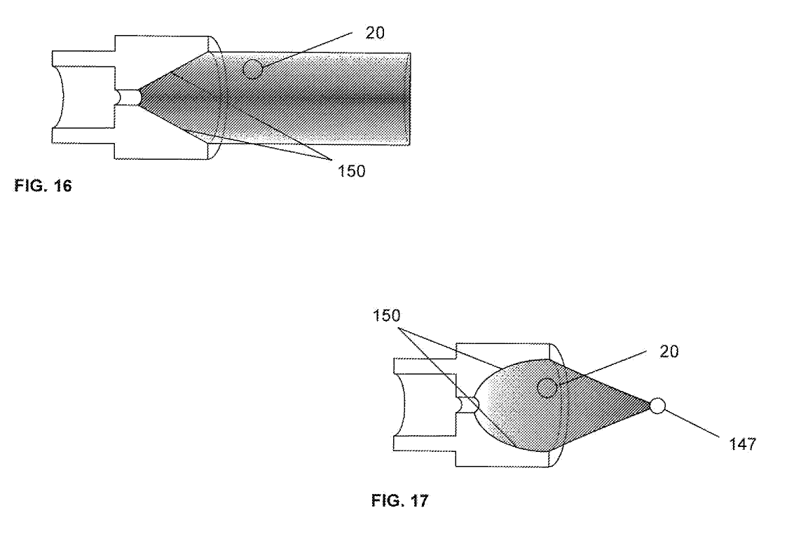

FIG. 16 is a cross-sectional view of an alternative embodiment of an ultrasound horn distal end including the radiation surface. The radiation surface 150 may also possess a conical portion as depicted in FIG. 16. Ultrasonic vibrations emanating from the conical portion direct the atomized spray inwards.

FIG. 17 is a cross-sectional view of an alternative embodiment of an ultrasound horn distal end including the radiation surface. The ultrasonic vibrations emanating from the concave portion of the radiation surface 150 depicted in focuses the spray of therapeutic solution 20 through focus 147. Maximizing the focusing of spray may be accomplished by constructing radiation surface 150 such that focus 147 is the focus of an overall parabolic configuration formed in at least two dimensions by a concave portion.

The radiation surface may possess any combination of the above mentioned configurations such as, but not limited to, an outer concave portion encircling an inner convex portion and/or an outer planar portion encompassing an inner conical portion.

Although specific embodiments and methods of use have been illustrated and described herein, it will be appreciated by those of ordinary skill in the art that any arrangement that is calculated to achieve the same purpose may be substituted for the specific embodiments and methods shown. It is to be understood that the above description is intended to be illustrative and not restrictive.

Combinations of the above embodiments and other embodiments as well as combinations of the above methods of use and other methods of use will be apparent to those having skill in the art upon review of the present disclosure. The scope of the present invention should be determined with reference to the appended claims, along with the full scope of equivalents to which such claims are entitled.

1. A topical therapeutic hyperbaric skin treatment system comprising:
an ultrasound station having an ultrasound horn with an internal chamber for mixing a therapeutic solution;
a matrix produced from at least the therapeutic solution;
a therapeutic pad containing at least the matrix; and
the therapeutic pad sealed within a storage container.

2. The system of claim 1 wherein the therapeutic solution includes oxygen and normal saline.

3. The system of claim 1 wherein the matrix includes a gauze component.

4. The system of claim 1 wherein the matrix includes a gel component.

5. The system of claim 1 wherein the matrix includes oxygen micro-bubbles some of which have a diameter of at least 10 microns.

6. The system of claim 1 wherein the therapeutic pad includes a fluid adsorption layer.

7. The system of claim 1 wherein the therapeutic pad includes a permeation layer.

8. The system of claim 1 wherein the therapeutic pad includes an adhesive layer.

9. The system of claim 1 wherein the therapeutic pad includes an impermeable layer.

10. The system of claim 1 wherein the therapeutic pad is stored within the storage container at a pressure between 5 and 50 psi.

11. The system of claim 1 wherein the ultrasound horn vibrates at a frequency between 15 kHz and 40 MHz.

12. The system of claim 1 wherein the ultrasound horn vibrates at a frequency at approximately 30 kHz.

13. The system of claim 1 wherein the ultrasound horn vibrates at an amplitude of greater than 1 micron.

14. The system of claim 1 wherein the ultrasound horn vibrates at an amplitude between 40 microns and 60 microns.

15. A method of applying oxygen to skin comprising the steps of:
a. mixing a fluid and a gas within an internal chamber of an ultrasound horn to form a therapeutic solution;
b. using the therapeutic solution to produce a matrix;
c. producing a therapeutic pad using at least the matrix;
d. sealing the therapeutic pad within a storage container;
e. treating a wound site by applying the therapeutic pad to the wound site and removing the therapeutic pad after a period of time.

16. The method of claim 15 wherein the therapeutic solution includes at least oxygen and normal saline.

17. The method of claim 15 wherein the therapeutic solution within the internal chamber includes oxygen micro-bubbles some of which have a diameter of at least 2 microns.

18. The method of claim 15 wherein the therapeutic solution forms a gel after exiting the ultrasound horn.

19. The method of claim 17 wherein the oxygen micro-bubbles are at least partially attached to strands within a matrix having a fibrous component.

20. The method of claim 17 wherein the oxygen micro-bubbles are at least partially entrapped within a matrix having a gel component.
